(19) United States
(12) Patent Application Publication
(10) Pub. No.: US 2005/0094823 A1
(43) Pub. Date: May 5, 2005

(54) Active noise controller and projector using the same

(76) Inventors: Tomoki Kobori, Kamakura (JP); Fumio Haruna, Yokohama (JP); Tsuneyuki Nozawa, Yokohama (JP)

Correspondence Address: ANTONELLI, TERRY, STOUT & KRAUS, LLP, 1300 NORTH SEVENTEENTH STREET, SUITE 1800, ARLINGTON, VA, 22209-9889, US

(21) Appl. No.: 10/824,431

(22) Filed: Apr. 15, 2004

(30) Foreign Application Priority Data
Oct. 29, 2003 (JP) 2003-368300

(51) Int. Cl. A61F 11/06; G10K 11/16; H03B 29/00

(52) U.S. Cl. 381/71.5; 381/71.3

(57) ABSTRACT

A low-cost and highly accurate active noise controller provides for low acoustic noise in a projector that makes produces highlight radiance and has a small size. The rotation frequency of a fan used for cooling the apparatus is measured by a rotation speed detector attached to the fan, a base frequency of acoustic noise and its multiple frequencies are calculated from the number of vanes provided on each fan, and target frequencies at which the acoustic noise is actively controlled are specified. An acoustic noise state is taken in from a microphone, a noise level is specified at each specified target frequency, the amplitude of an antiphase wave, that relates to the frequency characteristics of a microphone, a speaker and the inside of the active noise controller (or the projector), is roughly determined, and then a generated wave is emitted from the speaker. Further, for each target frequency, phase compensation is repeated in a time sequential manner, whereby the noise level is minimized.

### BACKGROUND OF THE INVENTION

This invention relates to an active noise controller for use in a household electric appliance or business-oriented appliance of the type in which the temperature inside it's the structure of the appliance is controlled by forced air flow generated by rotation of a cooling fan; and, the invention is directed in particular to a technique for reducing the acoustic noise of a cooling fan used in a projector having a high-power lamp.

In liquid crystal projectors, partly because the luminous energy of the lamp that is not available for use effectively appears mostly as heat, the inside of the apparatus becomes high in temperature, and, hence, it is indispensable to cool the interior of the apparatus. For this reason, cooling the inside of the apparatus is achieved typically by forced air flow generated by a cooling fan. At this point, it should be noted that, regarding the acoustic noise generated by such a cooling fan, low acoustic noise is achieved statically by modifying the shape of the fan, the rotation speed, and the method of driving the fan, or by optimizing the structural materials of the apparatus, the duct design, etc., under restrictions of the apparatus volume.

Further, as disclosed in JP-A Nos. 8581/1994 and 20866/1998, methods of achieving low acoustic noise dynamically—muffling the noise through an interference action—by detecting the acoustic noise generated by the cooling fan and generating an acoustic wave component having an antiphase waveform, have been proposed. In addition to this approach, a number of active noise control techniques using an antiphase wave have been proposed.

### SUMMARY OF THE INVENTION

In a liquid crystal projector, the demand for improvement toward obtaining a high luminance by the use of a high-power lamp and the provision of an apparatus of compact size have become main factors that disadvantageously increase the restrictions on the design of an air flow path thereon. In low acoustic noise in the conventional static technology, optimization of the design of an air flow path is conducted in order to maintain and improve the efficiency of a heat radiator, and the air flow rate is secured by increasing the number of cooling fans and increasing the speed of rotation of each cooling fan. However, there is a trade-off between the reduction of the acoustic noise and the performance of the projector, so that improvement in the luminance performance and size reduction in the size of the projector come with several problems, such as the occurrence of restrictions in the design.

On the other hand, the methods disclosed in the above-referenced patent publication are directed to active noise control that can be applied to only apparatuses that are cooled by a single cooling fan, and, further, variation in the speed of rotation of the cooling fan (speed drift) is not considered.

With a view toward improving the conventional technology, the object of this invention is to provide an active noise controller that realizes low acoustic noise at low cost with a high degree of accuracy, and a projector using this controller that achieves high luminance and has a compact size.

In order to solve the above-mentioned problems, this invention is directed to an active noise controller for an apparatus equipped with fans, each having a plurality of vanes, and a duct for guiding air from the fan, the controller comprising: a microphone for taking in the acoustic noise in the duct; rotation speed detecting means for detecting the fan rotation speed; frequency calculating means for calculating base and multiple frequencies based on the rotation speed and number of vanes of the fan; analyzing means for analyzing the acoustic noise that was taken in with the microphone in a time sequential manner for each of the base and multiple frequencies that were calculated by the frequency calculating means; phase controlling means for controlling the phase of the acoustic noise at each of the base and multiple frequencies in a time sequential manner; and signal generating means for generating a driving signal based on the analyzing means, the frequency calculating means, and the phase controlling means, wherein the active noise controller is configured to drive a speaker with the driving signal generated by the signal generating means.

This invention makes it possible to provide a low-priced, highly accurate active noise controller for use in a projector.

### BRIEF DESCRIPTION OF THE DRAWINGS

FIG. 1 is a block diagram of an active noise controller according to a first embodiment of the present invention;

FIG. 2 is a block diagram of an active noise controller for a projector according to this invention;

FIG. 3 is a timing chart that provides a supplementary illustration of first and second embodiments; and

FIG. 4 is a timing chart that provides a supplementary illustration of the first and second embodiments.

### DETAILED DESCRIPTION OF THE PREFERRED EMBODIMENTS

Hereafter, various embodiments according to this invention will be described with reference to the drawings.

[Embodiment 1]

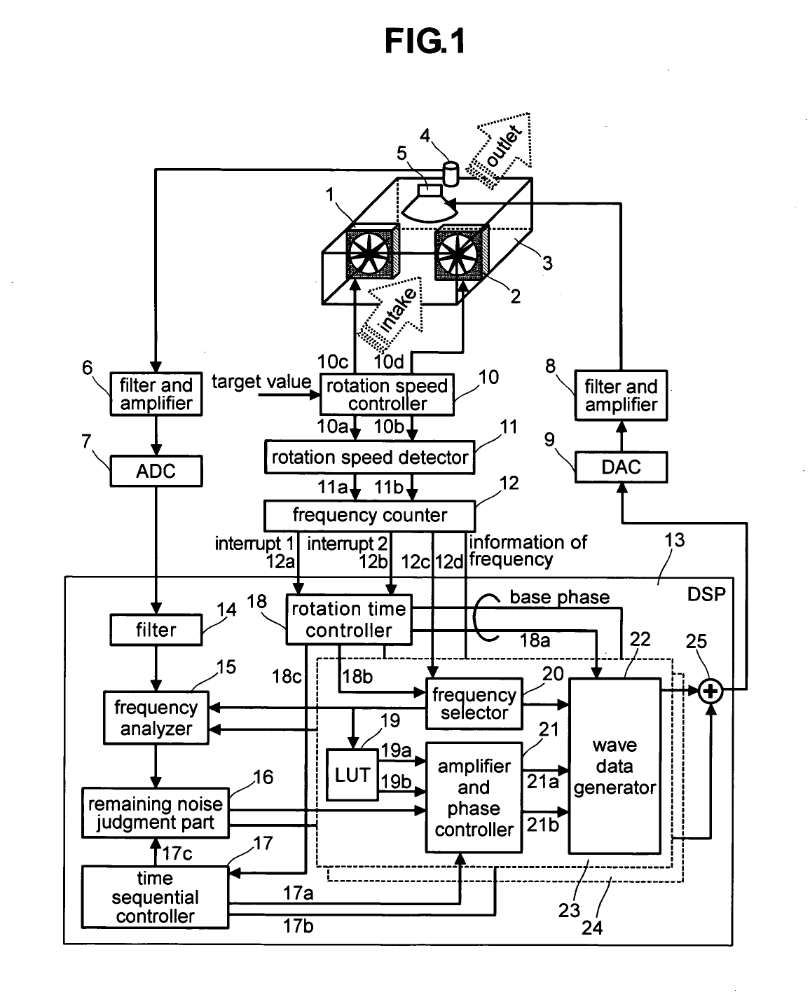

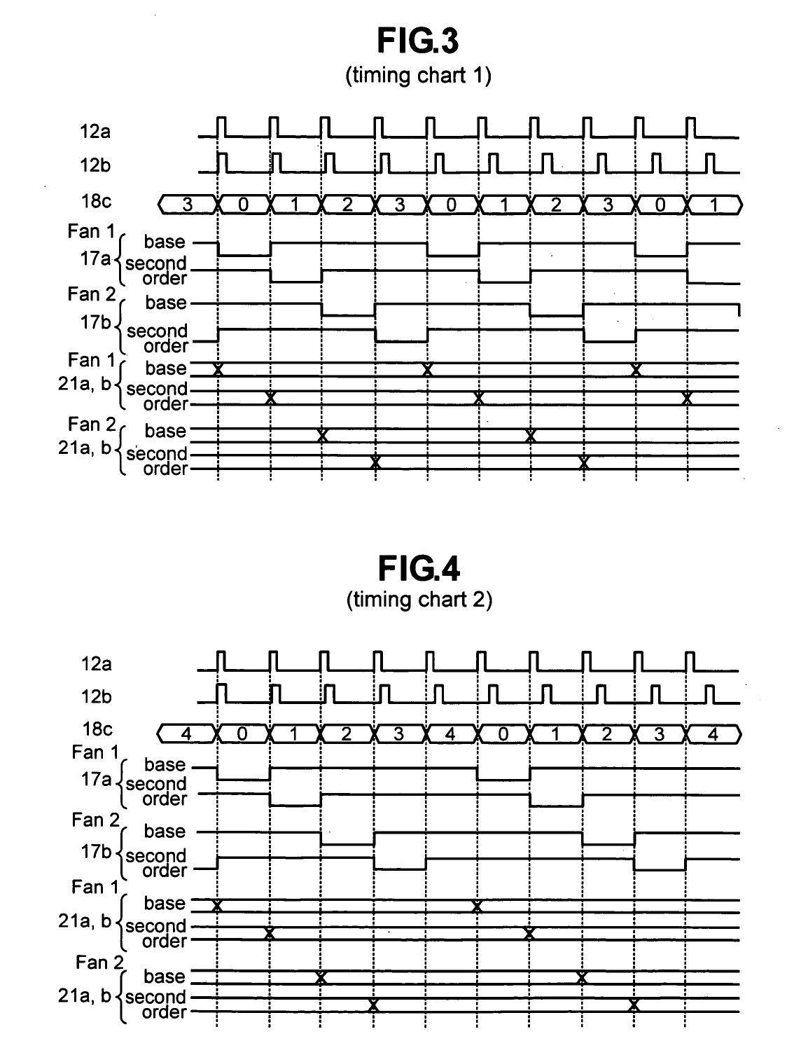

FIG. 1is a block diagram showing an example of the active noise controller according to this invention. FIGS. 3 and 4 are timing charts showing an outline of the operation of the present invention.

In FIG. 1, the invention is applied to a cooling unit including a pair of fans 1, 2, each having K number of vanes (e.g., K=7), and a duct structure 3 constituting the air flow path of cooling air. The controller for this cooling unit includes a microphone 4, a speaker 5, a filter and amplifier 6 for input compensation, an ADC (analog-digital-converter) 7 for converting an analog signal to a digital signal, a filter and amplifier 8 for output compensation, a DAC (digital-analog-converter) 9 for converting a digital signal to an analog signal, a rotation speed controller 10, a rotation speed detector 11, a frequency counter 12 for fan rotation, and a digital signal processor (referred to as a DSP) 13. The digital signal processor 13 includes a filter 14, a frequency analyzer 15, a remaining noise judgment part 16, a time sequential controller 17, a rotation time detector 18, a look-up-table (referred to as LUT) 19, is a frequency (f-value) selector 20, an amplifier and phase controller 21, a wave data generator 22, antiphase wave generator units 23, 24 each composed of constituents 19 to 22 for each fan, and an adder 25.

FIGS. 3 and 4 show the operation timing in each image-processing section.

Here, if the acoustic noise is observed while the fan 1 is being rotated, the amount of noise at specific frequency components is emphasized, as is well known. For example, when the fan makes M rotations (e.g., M=3000) for 1 minute, it is known that the noise base frequency NZ is determined by the following formula (formula 1) based on the rotation speed M and the number of vanes K.
NZ=M/60×K(=3000/60×7=350 Hz) (Formula 1)

When the rotation speed M=3000 and the number of vanes K=7, the noise base frequency is NZ=350 Hz, and the amount of noise at its multiple frequencies (NZ×2, ×3, ×4, . . . ) is emphasized. If the noise components are undesirable in the audible frequency band, they impact unpleasantly on the ear.

Hereafter, a method of actively controlling the noise base frequency NZ and its multiple frequencies that impact unpleasantly on the ear will be described in detail.

Note here that, in order to simplify the explanation of this embodiment, the example will be limited as follows: two identical fans each having seven vanes are used, and the target noise components of each fan are two components (the above-mentioned components NZ and NZ×2); therefore, a total of four frequency components are actively controlled. It is needless to say that a similar effect may be achieved even with a different number of vanes, a different number of fans, and where the number of target noise components is increased or decreased or the fan shape is modified. Moreover, the structure of the air flow path is shown to have a rectangular passage. It is needless to say that a similar effect may be achieved even if the apparatus to be targeted is configured to have an optimal structure.

In response to a rotation instruction (not shown in the figure), the rotation speed controller 10 controls the speed of rotation of the fan 1 to achieve the above-described rotation speed M and the speed of rotation of the fan 2 to achieve a rotation speed L with driving signals 10c, 10d, respectively. In this case, the rotation speed M of the fan 1 and the rotation speed L of the fan 2 are determined to have a relation M≠L so that any higher-order frequency component of the noise frequency that is determined by the information concerning frequency and the vane numbers will not be identical among the fans.

Rotation of the fans 1, 2 will generate air flow in the air flow path formed by the duct structure 3, and the microphone 4 monitors the acoustic noise state in the duct structure 3. Alternately, the processing may be carried out in such a way that the microphone 4 does not hinder the air flow. The microphone signal is subjected to simple noise reduction of low frequencies and high frequencies and various signal compensations, such as amplification of the signal level in the filter and amplifier 6, and it is then converted to a digital signal with a sampling frequency of Fs (Hz) by the ADC 7.

On the other hand, the DAC 9 converts a digital signal of an active noise controlling wave that was outputted from the adder 25 to an analog signal, which is subjected to removal of unnecessary frequency components and noises, and then is subjected to amplification in a filter and amplifier for output compensation 8, and this analog signal drives the speaker 5. A sound pressure vibration produced by the speaker 5 is emitted inside the duct structure 3. In this case, the active noise controller is configured so that the sound pressure vibration will not leak to the rear face of the speaker 5 (outside the duct structure 3).

The rotation speed detector 11 generates one-pulse/rotation signals 11a, 11b by the use of a Hall device signal, an FG signal, a photosensor signal, etc. as rotational information 10a, 10b of the fans 1, 2 to be used by the rotation speed controller 10. The frequency counter 12 outputs: an interrupt (1) 12a and an interrupt (2) 12b, that serve as reference timings in performing a calculation for phase adjustment in synchronization with the one-pulse/rotation signals 11a, 11b based on these signals; and information of the frequency 12c, 12d is obtained by measuring the rotation frequencies of the fans 1, 2.

The DSP 13 performs digital arithmetic processing, and will be described below with reference to the processing in each part on the assumption that it is a piece of hardware, in order to make the explanation easier to understand. In this embodiment, the DSP as shown is provided only as an example, but the present invention is not limited to this. Needless to say, any processor having the same processing function is applicable to this invention.

First, the filter 14 processes the digital signal received from the ADC 7 using a filter that assumes a filter characteristic for extracting signals in frequency bands of desired targets from the digital signal. The frequency analyzer 15 selects and extracts the amount of acoustic noise in the frequency components specified by a frequency selector 20, that will be described later, and determines a noise reduced level from a residual state of the amount of the acoustic noise obtained in the remaining noise judgment part 16. In this case, the remaining noise judgment part 16 is configured to prevent erroneous judgments caused by impulse disturbance resulting from the ambient circumstances (for example, the active noise controller may be affected by various kinds of daily life sounds, such as speech, a slapping sound, a clapping sound, the sound of a door opening and shutting). Specifically, in judging the noise reduced level by detecting the noise levels of the frequency components according to the time sequential control permission signal 17c received from the time sequential controller 17, the active noise controller is configured to perform a judgment considering the past noise levels of the frequency components. For example, the active noise controller calculates a histogram of the noise level, eliminates a range of level, except for the desired noise level, to get a modified noise level, and performs a judgment based on this level.

Using interrupts 12a, 12b as references, the rotation time detector 18 generates: a reference 18b of the rotation speed for each of the fans 1, 2; a base phase 18a of an active noise controlling wave for each of the fans 1, 2; and a time sequential timing signal 18c determined with respect to the counting of the rotation speed of the fan 1.

The time sequential controller 17 issues a control permission signal that indicates either control permission or control prohibition for each of the noise components (four components) of each fan with respect to the time sequential timing signal 18c in a time sequential manner (in timing diagrams 17a, 17b in FIGS. 3 and 4, LOW: permission period, HIGH: prohibition period). Here, since there is the possibility that the rotation phases of the two fans may agree with each other, the active noise controller is configured to perform a time sequential operation relative to one of the reference timings (12a) to which an operation for phase adjustment is performed. FIGS. 3 and 4 show an example in which a method of generation of the time sequential control permission signals 17a, 17b is changed. In this example, a permission period issuing method is not limited at all, but is defined to be optimal to the constituting conditions of a target frequency.

The LUT 19 takes in various kinds of frequency characteristics of the structure, the ambient environment, the speaker, and the microphone, and it outputs an amplitude compensation value 19a and a phase compensation value 19b that correspond to information of a target frequency to be actively controlled by the frequency selector 20.

The frequency selector 20 determines the base frequency (NZ) and the second-order frequency (NZ×2) that are governed by information of the frequency (=M/60) 12c, 12d and the number of vanes (K=7) of the fan. In determining these frequencies, since the fans 1, 2 rotate with some degree of rotational fluctuation (jitter), the frequency selector 20 may be configured to smooth the information of the frequency 12c, 12d relative to the counting of the rotation speed 18b.

The amplifier and phase controller 21 performs the following processing for each acoustic noise component of each fan. That is, in a period when the time sequential control permission signals 17a, 17b indicate a permission period, the amplifier and phase controller 21 controls the amplitude value and the phase shift quantity so that the amount of remaining noise determined by a judgment result of the remaining noise judgment part 16 becomes minimum. Then, the amplifier and phase controller 21 generates an amplitude value 21a and a phase shift quantity 21b that are the result of addition of the above-mentioned amplitude value and the amplitude compensation value 19a from the LUT 19 and the result of addition of the above-mentioned phase shift quantity and the phase compensation value 19b therefrom, respectively. On the other hand, in a prohibition period, the amplifier and phase controller 21 maintains and outputs the amplitude value 21a and the phase shift quantity 21b that were determined in the past permission period. Even in a period which is judged to be a prohibition period or in the case of a noise reduced level, if there is a change in the information of the frequency 12c, 12d, the amplitude value 21a and the phase shift quantity 21b may be compensated using an amplitude compensation value 19a and a phase compensation value 19b by the LUT 19.

Wave data generator 22 forms antiphase waves with respect to the fan noise components from items of information: four target frequencies of the fans 1, 2 obtained by the frequency selector 20, rotation base phase 18a, the amplitude value 21a, and the phase shift quantity 21b.

Here, the reference numerals 23, 24 denote antiphase wave generator units each composed of a LUT 19, a frequency selector 20, an amplifier and phase controller 21, and a wave data generator 22; and the active noise controller is shown to have of a configuration that includes as many units as the number of fans to be actively sound controlled (in this embodiment, two units). Needless to say, the number of the antiphase wave generator units is not limited to the number of fans; and, even when using as many antiphase wave generator units as target frequencies, a similar effect may be obtained.

The adder 25 adds four antiphase wave components obtained by the above-mentioned processing, and it outputs the addition result to the DAC 9.

The first embodiment is configured such that frequencies that impact unpleasantly on the ear are specified as target frequencies to be actively controlled, and the rotation speed is made to differ among a plurality of fans, thus simplifying the classification of acoustic noise states to and also simplifying the extraction of remaining noise components and the above-mentioned calculation. Thereby, it becomes possible to reduce the amount of calculation required by the DSP (digital-signal-processor), permitting selection of a low-priced DSP having a lower processing capability. Moreover, since the frequency components are specified, it becomes possible to simplify signal compensation of the microphone signal and the speaker driving signal. In addition to this, since high-grade and highly accurate analog parts are not required, it becomes possible to reduce the parts count and the parts cost.

The method employed is one that directly detects the rotation speed(s) and the rotation phase(s) of the fan(s). Thus, there is neither an increase in the amount of calculation, caused by continuous evaluation of a constantly measured noise level and a continuous following of the phase for detecting a fan rotation state, nor a delay in a follow-up control caused by delay in arithmetic processing. Further, there is no fear that the control will go into oscillation in some very extreme cases. Therefore, it is easily possible for the active noise controller to follow even the fan rotation jitter that occurs irregularly without going into oscillation.

Since the fan rotation speed and the rotation phase are directly detected, and, when generating the antiphase wave, it is done relative to the rotation phase of the fan, the frequency and the phase are fixed automatically by the antiphase wave and the fan rotation. Consequently, no special arithmetic processing is required.

The antiphase wave conditions are reexamined and used for control alternately in a time sequential manner. Thus, even when the target frequencies are increased, a needless increase in the peak calculation amount in the DSP may be prevented.

Since the target frequencies are specified, even if an impulse disturbance occurs, such a disturbance, except for the specified frequencies, is ignored.

Even if the frequency of the disturbance agrees with one of the specified frequencies, since the apparatus is configured to consider the past noise level of the frequency component, as described above, the disturbance is rejected. Thereby, it becomes easily possible to prevent oscillation of the antiphase wave caused by the disturbance.

In the consideration of this embodiment, a specific configuration is described. Needless to say, this invention is not limited to this embodiment, and certainly, this invention may be applied to a case where the location of the processing part, which was described to be either inside the DSP or outside the DSP, is changed.

[Embodiment 2]

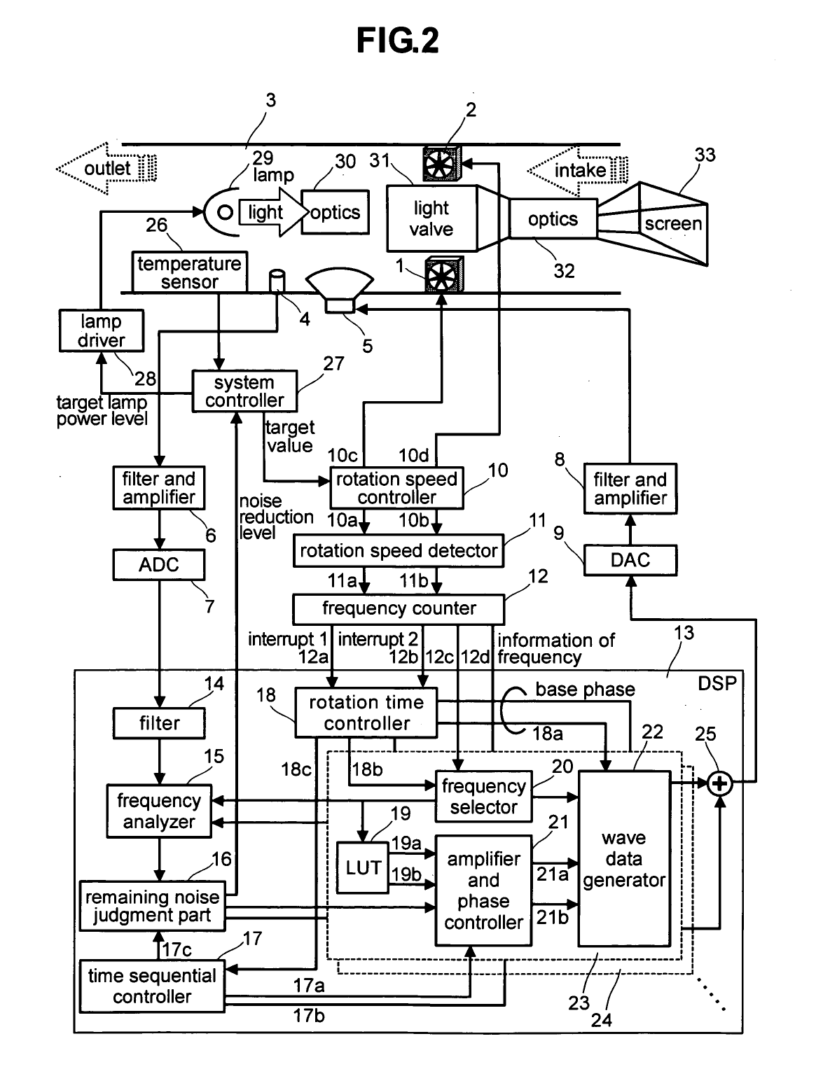

Next, the second embodiment of this invention will be described with reference to FIG. 2. Note that, since each part designated with the same reference numeral described in connection with the first embodiment has substantially the same function, a description thereof is omitted to avoid repeated description. In this embodiment, a liquid crystal projector is used as an example of image display apparatuses to which the present invention is applied.

In FIG. 2, there are a temperature sensor 26 for measuring the temperature inside the liquid crystal projector, a system controller 27 for controlling the system of the liquid crystal projector, a lamp driver 28, a lamp 29, optics 30, 32 composed of a lens, a filter, etc., respectively, a light valve 31, and a screen 33. The liquid crystal projector has a configuration such that parts of the lamp 29, the optics 30, the light valve 31, and the optics 32 are disposed inside the duct 3.

As a method of performing brightness adjustment of the projected image produced by the liquid crystal projector, there is a method of effecting an increase and a decrease in the amount of light of the lamp 29 by the lamp driver 28 increasing/decreasing the lamp driving electric power according to a target lamp power level received from the system controller 27. In this case, the amount of heat generation inside the duct 3 increases and decreases depending on the increase/decrease in the lamp driving electric power. Accordingly, the air flow rates necessary for controlling the temperature inside the duct 3 will fluctuate with time.

Thus, the liquid crystal projector needs to provide control in such a way that an optimal air flow rate may be obtained considering a trade-off between the acoustic noise and the air flow rate. For this purpose, for example, the temperature inside the projector is measured with the temperature sensor 26, and the system controller 27 controls the rotation speed controller 10, while controlling the electric power of the lamp driver 28, by issuing a rotation speed indication signal (target value) to the rotation speed controller 10 so that an optimal air flow rate is obtained.

In this case, the system controller 27 obtains information of a noise reduced level inside the liquid crystal projector from the remaining noise judgment part 16, and it controls the fan rotation speed so that it varies mildly within a range that the active noise control is able to follow.

According to the second embodiment, as described above, in an image display apparatus in which the speed of rotation of the cooling fan is controlled according to the temperature inside the apparatus, such as a liquid crystal projector, it becomes easily possible to realize brightness control by this method, while maintaining the noise reduced level, without generating oscillation even when the fan rotation speed is varied. This invention relates to a liquid crystal projector, and, thus, it may be employed for an image display apparatus, such as a low price consumer appliance, with the function of actively controlling noise without malfunction and oscillation regardless of several disturbance factors created by the surrounding environment, of the type described in connection with the first embodiment, such as of speech, sound caused by desk work, a clapping sound, and the sound of a door opening/shutting.

In the foregoing description, the embodiments are limited to a case where a plurality of fans are used, but this invention also may be applied to an apparatus with a single fan. In such a case, since the amount of processing is decreased, the number of controls may be increased or a low calculation amount DSP may be adopted, whereby coordinating the speed to the acoustic noise may be easily improved or the cost may be easily reduced, respectively.

Where a plurality of openings of the structure 3 is provided in an apparatus, a plurality of the same systems may be used. Alternatively, speakers and microphones may additionally be provided and the same processing may be performed in the DSP in parallel (in a time sequential manner). In either modification, a similar effect may be obtained.

An embodiment according to this invention was described in the case where its application was directed to a liquid crystal projector in a limited way. However, the application is not limited to this particular apparatus. A similar effect may be achieved in the case where this active noise controlling system is applied to an image display apparatus in which cooling inside the apparatus is carried by air flow generated by a fan or apparatus other than this. A similar effect may be realized with similar processing. For example, there are many applications, such as a refrigerator, an air cooling fan (indoor equipment/outdoor equipment), various kinds of engines, an air cleaner, a PC, and the like. On the other hand, it is easy to achieve even lower acoustic noise by using this in combination with a heat pipe, a liquid cooling system, etc.

According to this invention, by virtue of selection of a low-priced DSP because of a low calculation amount and reduction in analog parts count, a simple and low-priced active noise controller may be provided. Furthermore, in the projector in which the rotation speed of the cooing fan is controlled according to the temperature inside the apparatus, such as a liquid crystal projector, application of this invention enables the projector to have an active noise control effect free from malfunction and oscillation caused by following variations in the fan rotation speed and ambient disturbances, and this makes it possible to realize a low price which facilitates application for a consumer appliance.

Needless to say, this invention may be applied not only to an image display system, but also to other apparatuses that have cooling fans, and a similar effect of active noise control may be obtained by similar processing.

1. An active noise controller equipped with a fan having a plurality of vanes and a duct for guiding wind from the fan, comprising:
a microphone which takes in acoustic noise in the duct;
rotation speed detector which detects rotation speed of the fan;
frequency calculator which calculates base and multiple frequencies determined by said rotation speed and vane numbers of the fan;
analyzer which analyzes the level of the acoustic noise taken in with the microphone for each of the base and multiple frequencies calculated by the frequency calculator in a time sequential manner;
phase controller controls the phase of the acoustic noise for each of the base and multiple frequencies in a time sequential manner; and
signal generator which generates a driving signal based on the analyzer, the frequency calculator, and the phase controller,
wherein the active noise controller is configured to drive a speaker with the driving signal generated by said signal generator.

2. The active noise controller according to claim 1,
wherein the signal generator is configured to include adder which adds frequency signals of predetermined phases formed by the phase controller for each of the base and multiple frequencies.

3. The active noise controller according to claim 1,
wherein the phase controller is configured to control the signal generator so that the phase of a generated signal of each frequency corresponding to one of the base and multiple frequencies is shifted in order that a noise level analyzed at each of the base and multiple frequencies sent from the analyzer becomes smaller.

4. The active noise controller according to claim 1,
further comprising remaining noise discriminator which discriminates a noise reduced level based on a noise level at each of the base and multiple frequencies sent from the analyzing means,
wherein the active noise controller is configured to fix the amplitude value and the phase of the driving signal of a frequency at which the noise level is judged to be the noise reduced level.

5. The active noise controller according to claim 1,
further comprising a look-up-table which memorizes at least either of a phase compensation value corresponding to a variation in a frequency component of the acoustic noise or an amplitude compensation value corresponding to the frequency characteristic of said active noise controller,
wherein the active noise controller is configured to compensate the driving signal referring to said look-up-table.

6. The active noise controller according to claim 1,
further comprising a temperature sensor which detects the temperature inside the active noise controller; and
a rotation speed control circuit which controls the fan rotation speed,
wherein the active noise controller is configured to change and/or control the fan rotation speed based on temperature information from the temperature sensor and the remaining noise discriminator.

7. The active noise controller according to claim 1,
wherein the fans are composed of a plurality of fans, and
each fan differs from the other fans in a product of the vane numbers of the fan and the fan rotation speed.

8. A projector equipped with a lamp, an image display device for forming an optical image by modulating the intensity of light from said lamp, and projection lens which projects said optical image, comprising:
a cooling fan having a plurality of vanes and for cooling the lamp;
a duct which guides cooling air flow from said cooling fan to the lamp;
a temperature sensor which detects the temperature inside the projector;
a rotation speed control circuit which controls the rotation speed of the cooling fan;
a microphone which takes in acoustic noise in the duct;
rotation speed detector which detects the rotation speed of the cooling fan;
frequency calculator which calculates base and multiple frequencies that are based on said rotation speed and vane numbers of the fan;
analyzer which analyzes the level of the acoustic noise taken in with the microphone for each of the base and multiple frequencies calculated by the frequency calculator in a time sequential manner;
phase controller which controls the phase of the acoustic noise for each of the base and multiple frequencies in a time sequential manner; and
signal generator which generates a driving signal based on the analyzer, the frequency calculator, and the phase controller,
wherein the projector is configured to change and/or control the fan rotation speed according to temperature information from the temperature sensor, and drive a speaker with the driving signal generated by the signal generator.

9. The projector according to claim 8,
wherein the signal generator is configured to include adder which adds frequency signals of a predetermined phase formed by the phase controllor for each of the base and multiple frequencies.

10. The projector according to claim 8,
wherein the phase controller is configured to control the signal generator so that the phase of a generated signal of each frequency corresponding to one of the base and multiple frequencies is shifted in order that a noise level analyzed at each of the base and multiple frequencies sent from the analyzer becomes smaller.

11. The projector according to claim 8,
further comprising remaining noise discriminator which discriminates a noise reduced level based on a noise level at each of the base and multiple frequencies sent from the analyzer,
wherein the projector is configured to fix an amplitude value and a phase of the driving signal of a frequency at which the noise level was judged to be the noise reduced level.

12. The projector according to claim 8,
further comprising a look-up-table which memorizes at least either of a phase compensation value corresponding to a variation in a frequency component of the acoustic noise or an amplitude compensation value corresponding to the frequency characteristic of said projector,
wherein the projector is configured to compensate the driving signal referring to said look-up-table.

13. The projector according to claim 9,
wherein the phase controller is configured to control the signal generator so that the phase of a generated signal of each frequency corresponding to one of the base and multiple frequencies is shifted in order that a noise level analyzed at each of the base and multiple frequencies sent from the analyzer becomes smaller.

14. The projector according to claim 9,
further comprising remaining noise discriminator which discriminates a noise reduced level based on a noise level at each of the base and multiple frequencies sent from the analyzing means,
wherein the projector is configured to fix the amplitude value and the phase of the driving signal of the frequency at which the noise level was judged to be the noise reduced level.

15. The projector according to claim 9,
further comprising a look-up-table which memorizes at least either of a phase compensation value corresponding to variation in a frequency component of the acoustic noise or an amplitude compensation value corresponding to the frequency characteristic of said projector,
wherein the projector is configured to compensate the driving signal referring to said look-up-table.

16. The projector according to claim 13,
further comprising remaining noise discriminator which discriminates a noise reduced level based on a noise level at each of the base and multiple frequencies sent from the analyzing means,
wherein the projector is configured to fix the amplitude value and the phase of the driving signal of the frequency at which the noise level was judged to be the noise reduced level.

17. The projector according to claim 13,
further comprising a look-up-table which memorizes at least either of a phase compensation value corresponding to variation in a frequency component of the acoustic noise or an amplitude compensation value corresponding to the frequency characteristic of said projector,
wherein the projector is configured to compensate the driving signal referring to said look-up-table.
